(19) United States
(12) Patent Application Publication
(10) Pub. No.: US 2010/0030109 A1
(43) Pub. Date: Feb. 4, 2010

(54) DOUBLE-SIDE COATED SYRINGE NEEDLE

(76) Inventor: Wen-Hung LIN, Taipei City (TW)

Correspondence Address: LOWE, HAUPTMAN, HAM & BERNER, LLP (ITW), 1700 DIAGONAL ROAD, SUITE 300, ALEXANDRIA, VA, 22314, US

(21) Appl. No.: 12/490,468

(22) Filed: Jun. 24, 2009

(30) Foreign Application Priority Data
Aug. 1, 2008 (TW) 097129346

(51) Int. Cl. A61B 10/02; A61M 5/32

(52) U.S. Cl. 600/567; 604/272

(57) ABSTRACT

A hollow stainless syringe needle is coated with a layer of Zirconium or Zirconia to form a smooth inner surface and outer surface. With the Zirconium or Zirconia coated needle, a smooth wound shall be caused in human tissue. The human tissue shall recover more quickly for a smooth wound than an irregular one caused by a traditional stainless syringe needle. In addition, when used in human tissue biopsy, tissue's integrity and consistency can be maintained for different samples taken from the present invention, and thereafter test data comparison accuracy is ensured over the samples.

### RELATED APPLICATIONS

The present application is based on, and claims priority from, Taiwan Application Serial Number 097129346, filed Aug. 1, 2008, the disclosure of which is hereby incorporated by reference herein in its entirety.

### BACKGROUND

This disclosure relates to a double-side coated syringe needle, more particularly to a zirconium or zirconia coated syringe needle.

FIG. 1 shows a traditional syringe needle. A medical syringe needle 10 has a syringe needle body 11 and a hollow stainless steel needle head 12. The hollow needle head 12 is used to pierce into human being's skin tissue to inject liquid medicine into a human body or withdraw tissue samples from the hollow metal needle head.

FIG. 2 shows a second view along line AA′ of FIG. 1. The hollow metal needle head 12 has a hollow passage 122 and a rough surface 121 from a microcosmic view. Due to manufacturing process level and cost problem to a syringe needle 12, the roughness of the surface 121 of a traditional metal syringe needle head 10 is significant large enough to cause irregular tissue tear while piercing into human's body. The irregular tissue tear causes prolonged recover for a wound. In addition, a traditional syringe needle could make the wound tissue oxidation which causes the wound tissue being cured or repaired slowly by stem cells.

When hollow metal needle head 12 of the traditional syringe is used in human tissue biopsy, human tissue is taken out through the hollow passage 122. The known irregular and rough inner metal surface 124 tears the tissue, the sample integrity and consistency can not be maintained for different samples, and thereafter test data comparison accuracy can not be ensured over the samples. For a better medical study or observation over tissue samples, people need to get a smooth surface of syringe for tissue sampling to improve data accuracy of the samples.

### BRIEF DESCRIPTION OF THE DRAWINGS

FIG. 1 is a prior art

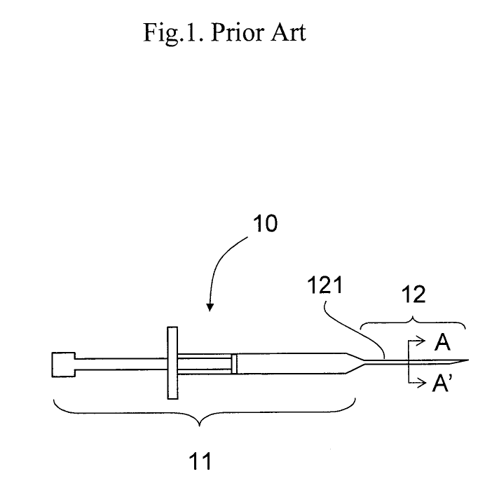

FIG. 2. is a section view of FIG. 1 along a section line AA′

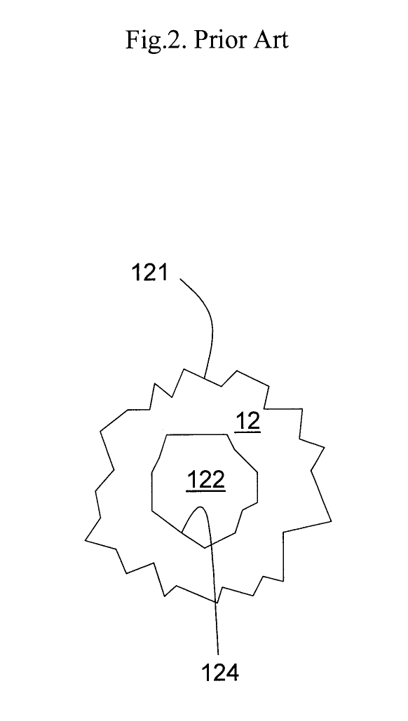

FIG. 3. is a first embodiment of the present invention

FIG. 4. is a second embodiment of the present invention.

### DETAILED DESCRIPTION OF EMBODIMENTS

The present invention overcomes the disadvantage of irregular tissue tear to which a traditional needle usually causes. According to this application, a syringe needle is coated with a smooth layer of zirconium or zirconia over both its inner surface and outer surface. The coating makes a hollow metal syringe needle to be with a smooth inner surface and outer surface.

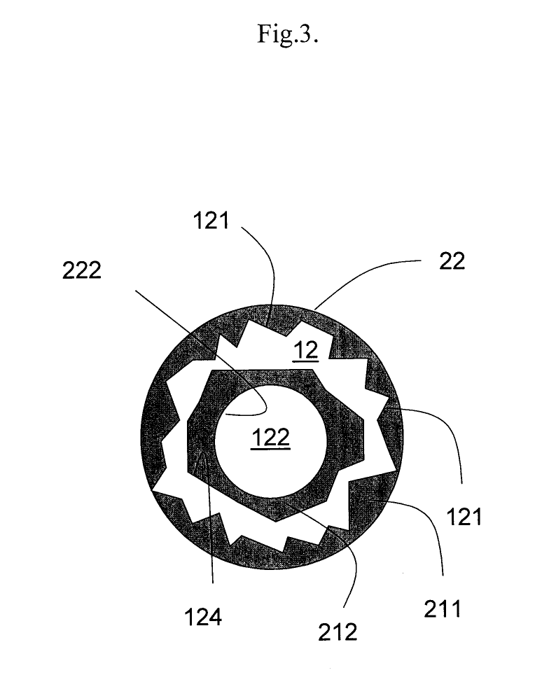

FIG. 3. is a first embodiment of the present invention. A section view of a double-side coated hollow metal needle head 12 according to the present invention shows a first smooth layer 211 with a thickness of 0.1˜1000 μm is applied over the outer rough surface 121 of the hollow metal needle head 12 to flat the roughness to yield an ideal outer smooth surface 22.

A second smooth layer 212 with a thickness of 0.1˜1000 μm is applied over the rough inner surface 124 of the hollow metal needle head 12 to flat the inner surface roughness to yield an ideal inner smooth surface 222. The central hollow 122 can be used to withdraw body tissues with perfect peripheral tissue for study or observation.

The outer smooth layer 211 is a material of zirconium or zirconia. The final outer smooth layer 211 can be made from vacuum plating or immersed chemical deposition to form a smooth surface 23 over the inner surface and outer surface of the hollow metal needle head 12.

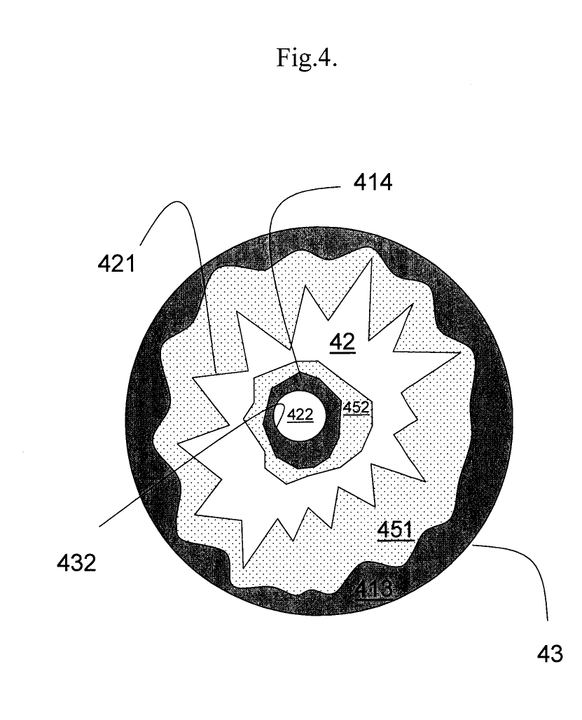

FIG. 4. is a second embodiment of the present invention. A first intermediate layer 451 is configured in between the hollow metal needle head 42 and an outer smooth layer 413. The outer intermediate layer 451 is used for flating a badly uneven outer surface 421. The outer intermediate layer 451 is applied to fill into the valleys to reduce the topography difference between valleys and peaks so as to provide a smoother outer surface of the hollow metal needle head 42 before applying a zirconium or zirconia fine layer 413 over the outer surface to yield a final smooth outer surface 43.

The material of the intermediate smooth layer 451 is selected from a material containing carbon content higher than that in a stainless steel. The intermediate smooth layer 451 also plays a role of a barrier layer for preventing carbon diffusion from the hollow stainless needle head 42 into the fine smooth layer 413. Carbon diffusion from the hollow metal needle head 42 might cause the smooth layer 413 deteriorated. The material of the intermediate layer 451 is selected from a group consisting of Teflon, PP, PE, PS, PET, PEEK, PMMA, and Nylon each of which has a thermal expansion coefficient⁺ between that of a stainless steel and zirconia. The coefficient is 10*10⁻⁶˜14*10⁻⁶/⁰ for a stainless steel, and 3*10⁻⁶˜9*10⁻⁶/⁰ for zirconia.

There are several methods which can be used for coating the intermediate layer 451 over the surface of the hollow metal needle head 42 such as immersed chemical deposition or die casting.

The final outer smooth layer 413 can be made through vacuum plating or immersed chemical deposition and forming a smooth outer surface 43 over the surface of the intermediate layer 451. Similarly, a second intermediate layer 452 is configured in between the hollow metal needle head 42 and the smooth layer 214 for yielding a smooth inner surface 432 surrounding a passing hole 422.

Each of the smooth layers 211,212,413,414 is exemplified as a material of zirconium or zirconia in the above embodiments, actually the material which can be used for the smooth layer 21,212,413,414 is one selected from a group consisting of: aluminum, silicon, silicon dioxide, aluminum oxide, titanium, titanium oxide, molybdenum, niobium, and tantalum.

While several embodiments have been described by way of example, it will be apparent to those skilled in the art that various modifications may be made without departing from the spirit of the present invention. Such modifications are all within the scope of the present invention, as defined by the appended claims.

What is claimed is:

1. A double-side coated syringe needle, comprising:
a hollow metal needle head, having an inner surface and an outer surface;
first ceramic layer, coated on the inner surface of the hollow metal; and
a second ceramic layer, coated on the outer surface of the hollow metal.

2. A needle as claimed in claim 1, further comprising:
a first intermediate layer, configured in between the hollow metal and the first ceramic layer; and
a second intermediate layer, configured in between the hollow metal and the second ceramic layer.

3. A needle as claimed in claim 1, wherein the ceramic layer comprising at least one material selected from a group consisting of: aluminum, aluminum oxide, silicon, silicon dioxide, zirconium, zirconia (zirconium oxide), titanium, titanium oxide, molybdenum, niobium, and tantalum.

4. A needle as claimed in claim 2, wherein the intermediate layer is a material selected from a group consisting of Teflon, PP, PE, PS, PET, PEEK, PMMA, and Nylon.

5. A needle as claimed in claim 2, wherein a thermal expansion coefficient for the intermediate layer is in the range between that of the hollow metal and zirconia.

6. A needle as claimed in claim 2, wherein a thermal expansion an coefficient for the intermediate layer is in the range between 10*10⁻⁶˜14*10⁻⁶/⁰ and 3*10⁻⁶18 9*10⁻⁶/⁰ depending on materials selected.
